(19) United States
(12) Patent Application Publication
(10) Pub. No.: US 2024/0099703 A1
(43) Pub. Date: Mar. 28, 2024

(54) MULTI-SENSOR CALIBRATION OF PORTABLE ULTRASOUND SYSTEM

(71) Applicant: X Development LLC, Mountain View, CA (US)

(72) Inventors: Alexander Martin Zoellner, Los Gatos, CA (US); Daniel Edward Rosenfeld, Mountain View, CA (US); Ashley Quinn Swartz, San Francisco, CA (US); John Paul Issa, San Francisco, CA (US); Joseph Hollis Sargent, San Francisco, CA (US); Ningrui Li, Palo Alto, CA (US); Phillip Yee, San Francisco, CA (US); Ulrich Niemann, Sunnyvale, CA (US); Qianyu Zhang, Mountain View, CA (US)

(21) Appl. No.: 18/373,179

(22) Filed: Sep. 26, 2023

Related U.S. Application Data
(60) Provisional application No. 63/409,903, filed on Sep. 26, 2022.

(51) Int. Cl. A61B 8/00

(52) U.S. Cl. CPC A61B 8/587

(57) ABSTRACT

This disclosure describes a system, method, and non-transitory computer readable media for an ultrasound probe configured to capture ultrasound images of an examination region. The system includes a first set of one or more sensors coupled to the ultrasound probe and configured to estimate a first positional information associated with the ultrasound probe. The system includes a second set of one or more sensors coupled to the ultrasound probe and configured to capture electromagnetic force (EMF) measurements in the examination region to estimate a second positional information associated with the ultrasound probe. The second positional information is used to calibrate the first set of one or more sensors. The system includes a controller configured to use at least one of (i) the first positional information, or (ii) the second positional information to generate a reconstruction of the examination region based on ultrasound images captured by the ultrasound probe.

### CROSS-REFERENCE TO RELATED APPLICATION

This application claims the benefit of U.S. Provisional Application No. 63/409,903 filed on Sep. 26, 2022, the entirety of which is herein incorporated by reference.

### TECHNICAL FIELD

This specification relates generally to medical devices, such as medical devices used for ultrasound imaging.

### BACKGROUND

Medical imaging devices, such as computed tomography scans and magnetic resonance imaging, provide medical providers with additional information to diagnose, monitor, and treat medical conditions. Medical imaging devices primarily recreate images or other types of representations to model parts of the body.

Conventional medical devices are expensive to operate and can provide measurements with poor fidelity, leading to inaccurate medical diagnoses and little insight in the growth or recovery of examined tissue. Poor measurement acquisition can be due to the inconsistent techniques applied from operator to operator. Medical devices provide a narrow perspective of examined tissue and often overlook other sensing technologies that can improve the overall fidelity of the acquired images. Additionally, the fidelity of the acquired data by a medical device is highly dependent on the operator of the medical device, which can range from a layperson with minimal training, and therefore low-cost for imaging, to a highly specialized medical professional with extensive training, and therefore a highly expensive cost for obtaining medical images.

Different types of sensors can become uncalibrated for a variety of reasons and degrade the accuracy of position estimations for medical devices. Estimating positions of medical devices is desirable, as the accuracy of position measurements for the medical device affects downstream processes such as image quality of images captured by the medical device. The reconstruction of an examination region based on the images can be degraded by poor estimates for medical device positions.

### SUMMARY

This specification describes an enhanced ultrasound system configured to improve ultrasound measurements by incorporating different types of sensors capturing different types of sensor data. For example, data from position sensors can be integrated with ultrasound information (and data from other sensors) to generate a representation of the examination area that is visually rich and conveys more information as compared to ultrasound information alone. Position sensors can be of various types each with its own advantages. For example, a mechanical position sensor such as a gyroscope can be used to estimate position/orientation information of a probe without significant computational burden. In some situations, however, such mechanical sensors can drift (i.e., lose calibration) thereby contributing to potentially inaccurate results. On the other hand, position sensors that are based on sensing an electromagnetic field (EMF) can be more robust to drift but pose a high computational burden for continuous real-time operations. The technology described herein allows for combining multiple different sensors—e.g., a mechanical position sensor and an EMF-based position sensor—such that a drift-prone sensor (or sensors) can be periodically calibrated using another sensor that is relatively more robust to drift—thereby providing for a balance between competing cost functions such as accuracy and computational burden.

By selecting a subset of data from the second set of sensors, the controller leverages the estimation from the second set of sensors to improve the accuracy of the first set of sensors. Thus, the controller of the ultrasound system is able to provide higher accuracy in positional estimates from the first set of sensors that are faster to process than processing the entire set of EMF measurements from the second set of sensors. By relying on EMF measurement-based positions for calibration only, the controller provides reduced consumption of computational resources compared to ultrasound systems that solely rely on processing EMF measurements, e.g., from the second set of sensors, to determine the position of the ultrasound system. Furthermore, the disclosed techniques and systems described in this specification may provide improved reliability as compared to ultrasound systems that solely rely on processing measurements from potentially drift-prone inertial measurement-based sensors, e.g., from the first set of sensors. Other types of sensors can be employed to improve calibration of the first set of sensors.

In an aspect, the subject matter described in this specification can be embodied in methods that include the actions of estimating, by a first set of one or more sensors coupled to an ultrasound probe, a first positional information associated with the ultrasound probe in an examination region; estimating, by a second set of one or more sensors coupled to the ultrasound probe, a second positional information associated with the ultrasound probe, wherein the second set of one or more sensors is configured to capture electromagnetic force (EMF) measurements in the examination region; calibrating, by a controller and using the second position information, the first set of one or more sensors; capturing, by the ultrasound probe, ultrasound images of the examination region; and generating, by the controller, a reconstruction of the examination region based on the ultrasound images and at least one of (i) the first positional information, or (ii) the second position information.

The foregoing and other embodiments can each optionally include one or more of the following features, alone or in combination.

In some implementations, the method includes capturing, by an additional set of one or more sensors, additional data of the examination region, and generating, by the controller, the reconstruction of the examination region based on the ultrasound images and the additional data.

In some implementations, calibrating the first set of one or more sensors includes adjusting a capture rate of at least one of (i) ultrasound images from the ultrasound probe, or (ii) positional information from a sensor in the first set of one or more sensors. In some implementations, a sensor in the first set of one or more sensors includes a gimbal or a gyroscope.

In some implementations, the method includes processing, by a model, the ultrasound images and at least one of (i) the first positional information, or (ii) the second positional information, and generating, by the model, the reconstruction of the examination region and a set of surface measurements associated with the examination region. In some implementations, the set of surface measurements includes at least one of (i) vessel stiffness, or (ii) vessel diameter, of the examination region. In some implementations, the model is at least one of (i) a machine learning network, (ii) a physical simulation, or (iii) a computational model.

In some implementations, calibrating the first set of one or more sensors includes determining a difference between the first positional information and the second positional information.

Other implementations of this and other aspects include corresponding systems, apparatus, and computer programs, configured to perform the actions of the methods, encoded on computer storage devices. A system of one or more computers can be so configured by virtue of software, firmware, hardware, or a combination of them installed on the system that in operation causes the system to perform the actions. One or more computer programs can be so configured by virtue of having instructions that, when executed by data processing apparatus, cause the apparatus to perform the actions.

In an aspect, a non-transitory computer-readable medium storing one or more instructions executable by a computer system to perform operations of estimating, by a first set of one or more sensors coupled to an ultrasound probe, a first positional information associated with the ultrasound probe in an examination region; estimating, by a second set of one or more sensors coupled to the ultrasound probe, a second positional information associated with the ultrasound probe, wherein the second set of one or more sensors is configured to capture electromagnetic force (EMF) measurements in the examination region; calibrating, by a controller and using the second position information, the first set of one or more sensors; capturing, by the ultrasound probe, ultrasound images of the examination region; and generating, by the controller, a reconstruction of the examination region based on the ultrasound images and at least one of (i) the first positional information, or (ii) the second position information.

In an aspect, an ultrasound system can include an ultrasound probe configured to capture ultrasound images of an examination region; a first set of one or more sensors coupled to the ultrasound probe and configured to estimate a first positional information associated with the ultrasound probe; a second set of one or more sensors coupled to the ultrasound probe and configured to capture electromagnetic force (EMF) measurements in the examination region to estimate a second positional information associated with the ultrasound probe, wherein second positional information is used to calibrate the first set of one or more sensors; and a controller configured to use at least one of (i) the first positional information, or (ii) the second positional information to generate a reconstruction of the examination region based on ultrasound images captured by the ultrasound probe.

In some implementations, the first positional information includes at least one of (i) a position, or (ii) an orientation, of the ultrasound probe. In some implementations, the second positional information includes at least one of (i) a position, or (ii) an orientation, of the ultrasound probe.

In some implementations, calibrating the first set of one or more sensors includes adjusting a capture rate of at least one of (i) ultrasound images from the ultrasound probe, (ii) a sensor from the first set of one or more sensors, or (iii) a sensor from the second set of one or more sensors, based on at least one of (i) the first positional information, or (ii) the second positional information. In some implementations, a sensor in the first set of one or more sensors includes a gimbal or a gyroscope.

In some implementations, the system includes a computing device communicatively coupled to the controller, wherein the computing device is configured to perform operations including: processing, by a model, the ultrasound images and at least one of (i) the first positional information, or (ii) the second positional information; and generating, by the model, the reconstruction of the examination region and a set of surface measurements associated with the examination region. In some implementations, the set of surface measurements includes at least one of (i) vessel stiffness, or (ii) vessel diameter, of the examination region. In some implementations, the model is at least one of (i) a machine learning network, (ii) a physical simulation, or (iii) a computational model.

In some implementations, the system includes an additional set of one of more sensors coupled to the ultrasound probe, the additional set of one or more sensors configured to capture additional data of the examination region; wherein the controller is configured to generate the reconstruction of the examination region based on the ultrasound images and the additional data.

In some implementations, a sensor in the additional set of one or more sensors includes a pressure sensor configured to capture force measurements of the examination region.

In some implementations, a sensor in the additional set of one or more sensors includes a camera device configured to capture one or more images of the examination region. A second sensor in the additional set of sensors includes a structured light source configured to illuminate at least a portion of the examination region, wherein the camera device is configured to capture one or more images of the examination region while the portion of the examination region is illuminated.

In some implementations, the additional set of one or more sensors includes a plurality of peripheral sensors, wherein a peripheral sensor from the plurality of peripheral sensors is configured to capture surface measurements of the examination region. The peripheral sensor can be a skin resistance sensor configured to capture skin resistivity measurements of the examination region. The peripheral sensor can be a pulse oximeter configured to capture pulse rate measurements of the examination region. The peripheral sensor can be a photoplethysmography sensor configured to capture at least one of (i) pulse rate measurement or (ii) blood pressure measurement of the examination region.

In some implementations, the plurality of peripheral sensors includes a light emitting diode configured to emit a pattern of light on at least a portion of the examination region; and a light detector configured to determine a deformation of the pattern of light on the portion of the examination region. The additional data of the peripheral sensor includes depth measurements representing the deformation of the portion of the examination region.

In some implementations, the second set of one or more sensors includes a pair of sensors. A first sensor in the pair of sensors can be positioned onto a surface of the examination region. A second sensor in the pair of sensors can be mounted onto the ultrasound probe. The controller can be configured to determine an amount of distortion based on a first set of EMF measurements from the first sensor and a second set of EMF measurements from the second sensor.

In some implementations, the system includes a computing device configured to generate, by a model, a multi-dimensional representation of the examination region based on the ultrasound images and at least one of (i) the first positional information or (ii) the second positional information. The multi-dimensional representation includes the reconstruction of the examination region and associated measurements of the examination region. The associated measurements are captured by at least one of (i) the first set of one or more sensors, or (ii) the second set of one or more sensors, the computing device can be communicatively coupled to the controller, and the model is at least one of (i) a machine learning model, (ii) a physical simulation, or (iii) a computational model.

Particular embodiments of the subject matter described in this specification can be implemented so as to realize one or more of the following advantages.

The ultrasound device described in this specification incorporates multiple types of sensor data from one or more sensors to improve the accuracy of measurements acquired in ultrasound imaging. The ultrasound device directly fuses the different types of data from multiple sensors and cameras using a computing model to generate a high-fidelity reconstruction of the imaged object e.g., tissue, appendage, bone. The ultrasound device also provides strict, consistent repeatability of the system using sensor feedback, by assisting the user to dynamically adjust the method of data acquisition while performing imaging with the ultrasound device. The ultrasound device records the pose of the device while in use, determines an ideal path for an operator to follow when acquiring medical data, and provides user feedback to ensure the operator follows the ideal path each time data is collected. The ultrasound device also uses the computing model to provide feedback to the operator on locations or poses for additional data collection.

The ultrasound device of the present disclosure employs a computing model, which may rely on physical simulation, machine learning, or advances in computing technology to construct highly accurate mappings between the measured sensor data and a reconstruction of the imaged object. The computing model of the ultrasound data directly fuses data from the sensors and predicts progression of future images of the imaged object e.g., the spread of disease in muscle tissue or monitoring the recovery of tissue. The computing model of the ultrasound data uses the current data in combination with past screening data to detect changes in tissue shape, density, volume, etc. The reconstructions can be generated without the loss in quality during measurement acquisition, as the ultrasound device employs a gimbal with path-planning and tracking to ensure consistent, repeatable measurements of the imaged object, e.g., muscle tissue. The reconstructions can also provide significant amounts of information to diagnose or track the progress of the imaged tissue by associating measurements from sensors observing the tissue with the ultrasound images of the tissues.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1A illustrates an example ultrasound system that includes multiple sets of sensors.

FIG. 1B illustrates an example ultrasound probe capturing ultrasound images.

FIG. 2 illustrates an example process for calibrating the ultrasound system.

FIG. 3 illustrates an example computing device for calibrating the ultrasound system using the multiple sets of sensors.

Like reference numbers and designations in the various drawings indicate like elements.

### DETAILED DESCRIPTION

Ultrasound systems are utilized by medical professionals to capture ultrasound images of an examination region of a patient, e.g., a patient's abdomen. An ultrasound system can be coupled to multiple types of sensors to improve the image quality of ultrasound images, and additional types of data can improve reconstruction of the examination region, e.g., including surface measurements of the imaged region. However, different types of sensors can have varying computational demands and become uncalibrated. As an example, a gyroscope can estimate angles and angular velocity of ultrasound probes, but experience gyroscope drift, e.g., noise from gyroscope components causing instability in angular measurements. As another example, electromagnetic tracking systems can be used to track positions of an ultrasound probe but can be computationally expensive to process and sensitive to distortion, e.g., metallic objects causing fluctuations in electromagnetic field measurements.

A controller of the ultrasound system can determine differences between positional estimates from different types of sensors, e.g., the first set of sensors and the second set of sensors. Based on the difference in positional estimates, the controller calibrates the first set of sensors to improve the accuracy of positional estimates from the first set of sensors. To calibrate the first set of sensors, the controller can determine a corresponding sample rate or time instance to utilize position estimates from the second set of sensors to adjust operation of the first set of sensors.

Calibrating a first set of sensors can improve position estimates of the ultrasound probe, thereby improving the quality of ultrasound images captured for an examination region. The first set of sensors capture sensor measurements using a mechanical-based type of sensor such as gyroscopes and gimbals. Other types of inertial movement-based sensors used for position estimation can achieve a high capture rate for sensor measurements and can be processed faster than electromagnetic force measurements but are sensitive to noise. A second set of sensors includes electromagnetic force measurements that can provide higher data fidelity for position estimates than the first set of sensors, but data capture rates for the EMF-based sensors are sensitive to distortion, e.g., from metal objects in an environment for an ultrasound system.

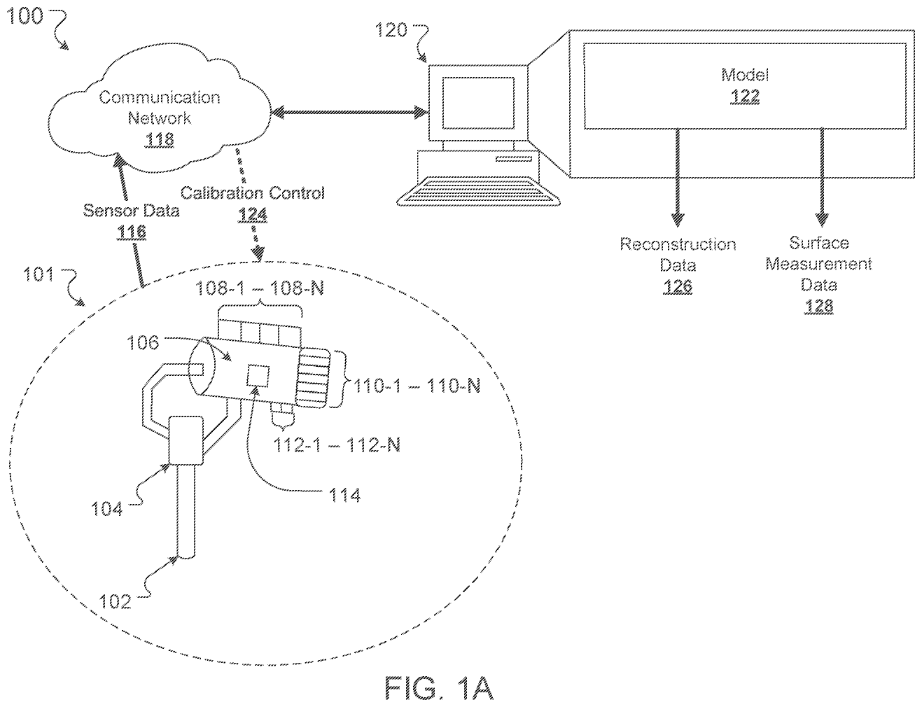

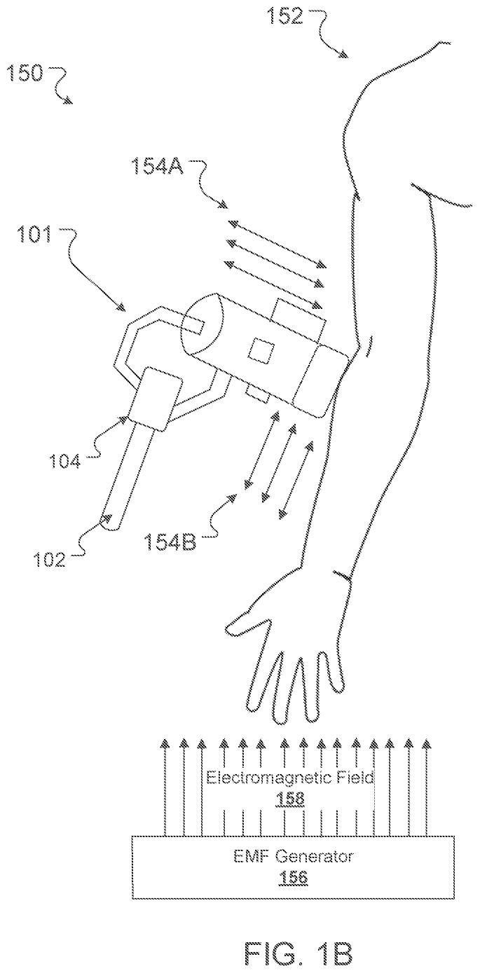

FIG. 1A shows an example ultrasound system 100 with multiple sets of sensors to improve the accuracy of position estimation for an ultrasound probe, by calibrating a first set of sensors based on positional estimates from another set of sensors. The ultrasound system 100 includes an ultrasound probe 101 configured to capture ultrasound images of an examination region of a patient. The ultrasound probe 101 is coupled to a computing device 120 by a communication network 118 and can be configured to provide sensor data 116 to the computing device 120. The computing device 120 can utilize the model 122 to process the sensor data 116 and generate reconstruction data 126 and surface measurement data 128. The reconstruction data 126 can include a multi-dimensional representation of an examination region and the surface measurement data 128 can include surface characteristics from the examination region such as blood vessel diameter and vessel stiffness.

The ultrasound probe 101 includes a handle 102 where the ultrasound device 100 can be held by a user, e.g., a medical professional. In some implementations, the handle 102 of the ultrasound probe 101 may include additional components e.g., buttons and triggers to operate the ultrasound probe 101 by enabling and configuring sensors.

The ultrasound probe 101 is connected to the handle 102 by a gimbal 104, e.g., a pivoted support that includes sensors and motors to stabilize the ultrasound probe 101. The ultrasound probe 101 includes a controller 114 configured to calibrate sensors of the ultrasound probe 108, by estimating positional information based on sensor measurements from a first set of one or more sensors 108-1-108-N (collectively referred to as “first set of sensors 108”) that are coupled to an ultrasound probe body 106 of the ultrasound probe 101. The first set of sensors 108 are configured to capture sensor measurements that indicate positional information about the ultrasound probe based on the gimbal 104, such as position and orientation of the ultrasound probe. The first set of sensors 108 can be referred to as positional sensors of the gimbal 104.

The ultrasound probe 101 includes a second set of one or more sensors 110-1-110-N, (collectively referred to as “second set of sensors 110”), that are depicted in FIG. 1A at the tip of ultrasound probe 101, e.g., a portion of the ultrasound probe that comes into contact with an examination region to capture ultrasound images. The second set of sensors 110 capture sensor measurements indicating positional information about the ultrasound probe, in which the sensor measurements are electromagnetic force (EMF) measurements of the examination region.

The ultrasound probe 101 also includes an additional set of one or more sensors 112-1-112-N (collectively referred to as “additional set of sensors 112”) that are depicted as being mounted onto the ultrasound probe body 106. In some implementations, the additional set of sensors 112 can include peripheral sensors configured to capture surface measurements of the examination region. Examples of peripheral sensors can include cameras, light emitting diodes coupled to a light emitting diode, and structured light sources. Additional sensor data from the additional set of sensors 112 can be provided to the controller 114 to calibrate the capture rate of the first set of sensors 108.

The controller 114 is configured to determine and adjust the capture rates of the sensors of the ultrasound probe, e.g., the first set of sensors 108, the second set of sensors 110, the additional set of sensors 112, or some combination therein, to calibrate the first set of sensors 108. As an example, the positional information estimated from the first set of sensors 108 can include measurements from a gyroscope, inertial measurement unit, etc. in the first set of sensors 108 captured at first capture rate, e.g., 1 kHz or 1000 samples per second. The controller 114 can determine that the first set of sensors 108 is uncalibrated, e.g., due to gyroscopic drift, by estimating positional information from the second set of sensors 110 that are based on EMF measurements. The second set of sensors 110 can capture EMF measurements at a second capture rate, e.g., 100 Hz or 100 samples per second, in which the controller determines a time instance to process the EMF measurements to estimate a second position information for the ultrasound probe 101. The second capture rate for the EMF measurements of the second set of sensors 110 can be lower due to distortion in the environment, e.g., in an environment of the ultrasound system 100, as the distortion renders fewer frames of measurements to be used for position estimation.

The controller 114 calibrates the first set of sensors 110 from positional information estimated by the EMF measurements from the second set of sensors 110. For example, the controller 114 determines a difference between the estimated position for the ultrasound probe from a first estimate determined from gyroscope measurements, e.g., a sensor in the first set of one or more sensors, and a second estimate determined from EMF measurements, e.g., an EMF detector in the second set of one or more sensors. The controller 114 can provide an instruction to a sensor from the first set of sensors 108, in which the sensor performs one or more calibration processes until the positional estimate from the first set of sensors 108 matches the positional estimate from the EMF measurements of the second set of sensors 110, e.g., within a threshold value. In some implementations, the controller 114 performs the calibration by computing an offset for one or more axes of a sensor (e.g., a sensor) in the first set of sensors 108 and compensates measurement readings from the sensor by the offsets.

In some implementations, the controller 114 adjusts the operation of a sensor in the first set of sensors by adjusting a capture rate. Adjusting the operation of the sensor can include increasing the capture rate to capture additional measurements for position estimation while the sensor is calibrated, e.g., within a threshold calibration range. By increasing the capture rate while the sensor is within a calibrated range, the controller 114 can achieve improved accuracy in positional estimates as data quality falls within a calibrated range of operation for the sensor, e.g., in the first set of one or more sensors. As another example, the controller can adjust operation of the sensor by decreasing the capture rate to capture fewer measurements for position estimation while the sensor is uncalibrated, e.g., outside the threshold calibration range. By reducing the capture rate while the sensor is outside a calibrated range, fewer poor-quality measurements can be processed, thereby reducing computational demands for the ultrasound system. Furthermore, a reduction of capture rate while measurement quality is poor mitigates accuracy loss in the image, compared to ultrasound images that capture an extraneous number of poor-quality measurements.

Although the controller 114 can be configured to process EMF measurements and estimate the position of the ultrasound probe 101 solely using the second set of sensors, any source of EMF distortion in an environment of the ultrasound probe 101 can result in a lower frame rate of EMF measurements, e.g., fewer EMF measurements detected. The disclosed ultrasound system 100 can be configured to rely on the lower frame rate of EMF measurements to calibrate one or more sensors in the first set of sensors, in which the estimation is a mechanics-based and less computationally expensive to estimate the position of the ultrasound probe 101. The controller 114 improves the estimation of the position and orientation for ultrasound probe 101, thereby improving the quality of ultrasound images captured by the ultrasound probe 101.

In addition to the controller 114, the computing device 120 can be coupled to the ultrasound probe 101 to process sensor data 116 and generate calibration control 124 for the sensors of the ultrasound probe 101. The computing device includes the model 122, which can determine adjustments, e.g., a capture rate, a configuration, for one or more sensors among the first set of sensors 108. Similar to the controller 114, the calibration for the sensors determined by the model 122 is based on the EMF measurements from the second set of sensors 110. The model 122 is configured to generate reconstruction data 126, e.g., a multi-dimensional representation of the examination region and the surface measurement data 128, e.g., surface characteristics from the examination region. The model 122 can be a machine learning model trained to one or more outputs of the computing device 120. In some implementations, the model 122 can also be a computational model, a physical simulation, a machine learning network, or some combination therein.

As an example, the model 122 can reconstruct a surface representation or multi-dimensional representations of the examination region of patient 152 by combining measurements (e.g., from the first set of sensors 108), structured light measurements, stereo photography, and force/pressure measurements. The structured light measurements, images, and force/pressure measurements can be captured by additional sensors of the ultrasound probe 101. In some examples, the model 122 can be trained to predict future measurement data from the measurements of sensors in the ultrasound system 100. The future measurement data can describe the progression (e.g., recovery, spread of disease) of the examination region.

The model 122 can perform a variety of training techniques to improve pose estimation of ultrasound probes in an ultrasound imaging environment, including supervised and unsupervised learning. In some examples, the model 122 performs hybrid-learning techniques to improve ultrasound imaging. The training of the model 122 can be performed using obtained ground truth data that includes ultrasound images with known positional information of the ultrasound probe. The model 122 can adjust one or more weights or parameters to match estimates or predictions from the model to the ground truth data. In some implementations, the model 122 includes one or more fully or partially connected layers. Each of the layers can include one or more parameter values indicating an output of the layers.

To improve data capture of the ultrasound images, the ultrasound probe 101 includes a gimbal 104. The gimbal 104 can include gyroscopes, inertial measurement units, or other types of sensors to provide additional stability while operating the ultrasound probe 101. The gimbal 104 improves the fidelity of data capture by sensors e.g., the first set of sensors 108, the second set of sensors 110, the additional set of sensors 112, by implementing stability, motion-tracking, and route-planning features. In some implementations, additional sensor data from the additional sensors 112 can also be used to calibrate positional information from the first set of sensors, e.g., angular measurements from a sensor.

The controller 114 can utilize feedback from the gimbal 104 of the ultrasound probe 101 and stabilize an angle of orientation for the ultrasound probe, e.g., while the ultrasound probe 101 captures images of the examination region. For example, the ultrasound probe 101 can be stabilized as the tip of the ultrasound probe is in contact with a surface of the examination region. The controller 114 can leverage stabilization hardware and software from the gimbal to stabilize the ultrasound body 106 and calibrate the first set of sensors 108, based on positional measurements from the second set of sensors 110. In some implementations, the ultrasound probe 101 utilizes a gyroscope 104 to provide stabilization along a single axis, in contrast to stabilization in multiple axes provided by a gimbal, e.g., roll, pitch, and yaw.

Although the first set of sensors 108 is depicted in FIG. 1A as being mounted onto an outer surface of the ultrasound probe body 106, the first set of sensors 108 can be mounted at the tip of the ultrasound probe, e.g., similar to the depiction of the second set of sensors 110. The second set of sensors 110 can also be mounted onto the surface of the ultrasound probe body 106, e.g., similar to the depiction of the first set of sensors 108. In some implementations, any of the sets of sensors (e.g., the first set of sensors 108, the second set of sensors 110, the additional set of sensors 112) are internally enclosed by the ultrasound probe body 106. In some implementations, the ultrasound probe body 106 can include a display (not illustrated) to provide real-time feedback, analysis, or other types of information to a user of the ultrasound probe 101 as medical data e.g., ultrasound images, sensor measurements, is acquired.

Referring to FIG. 1B, the ultrasound probe 101 can be used in an environment 150 to acquire medical data including measurements and images from a patient e.g., patient 152, by applying the ultrasound probe 101 to an appendage (e.g., arm, leg) or body part (e.g., abdomen) to be examined. The ultrasound probe 101 is configured to be adjusted when examining a patient e.g., by moving the ultrasound probe 101 towards or away from the patient 152 in directions 154A or following a path along the surface of the patient in directions 154B. The ultrasound probe 101 can be used to acquire medical data through measurements e.g., from the first set of sensors 108, the second set of sensors 110, the additional set of sensors 112, or some combination therein.

The measurements of an examination region of the patient 152 can characterize medical conditions or characteristics of the patient 152. An examination region includes a physical location on the surface of the body of patient 152, e.g., an appendage. In some implementations, the examination region includes the surface of the body of patient 152 that includes multiple organs to be imaged, e.g., an abdomen can be examined to analyze deformations of muscle tissue. In some implementations, ultrasound images can be utilized to examine deformations of organs, e.g., intestines, kidneys, liver. For example, the ultrasound device 110 can acquire measurements related to the examination region of the patient using sensors to generate a virtual representation of the examination region using acquired measurements, e.g., by the computer device 120.

The environment 150 also includes an EMF generator 156 configured to generate an electromagnetic (EM) field 158 in the environment 150. The EMF generator 156 provides the EM field 158, in which the second set of sensors 110 can be configured to measure the EM field 158, which can include a particular shape and/or intensity. The second set of sensors 110 capture EM measurements and can estimate position of the ultrasound probe 101, e.g., relative to the EM field.

Sensors of the ultrasound probe 106 may can characteristics of an examination region of patient 152 to determine or diagnose illnesses or aid in recovery. As an example, the gimbal 104 can adjust the angle of approach when a user applies the ultrasound probe 101 to an examination region of patient 152 to improve the quality of the acquired measurements. Adjusting the angle of approach of a sensor e.g., direct contact with a sensor on the tip of the ultrasound probe 101 can improve the quality of measurements by increasing the surface area of contact between the sensor at the tip and the examination region of patient 152. As an example, the gimbal 104 can adjust the orientation of ultrasound probe 101 to guide the user to acquire a series of measurements e.g., in a direction 154B in a desired path on the examination region of patient 152.

The ultrasound probe 101 can be coupled with memory storage and processing instructions, e.g., local memory, memory on computing device 120, to store the desired path, which may be used for a repeated series of measurements following the desired path. The repeated series of measurements following the desired path for future measurements using the ultrasound probe 101, which can be configured to repeat the desired path and guide the user with user feedback. As an example, the gimbal 104 provides stability support for highly repeatable high-fidelity measurements to track changes in the examination region (e.g., recovery of broken bones, sizes and spread of tumors) of a patient 152.

The ultrasound probe 101 captures medical images by transmitting electrical signals e.g., sound waves by a transducer, through an examination region (e.g., appendage, body part) of a patient 152, and receiving a set of return signals (e.g., frequency-shifted waves) reflected from boundaries within the examination region. As an example, the ultrasound probe 101 can determine boundaries between tissue and bone, or soft tissue and fluid, or some combination therein. In some implementations, the ultrasound probe 101 of ultrasound system 100 can transmit signals in various configurations, e.g., linear, curvilinear, phased array, and endocavitary.

The ultrasound probe 101 can provide an internal geometry of the examination region of a patient 152 by acquiring measurement data e.g., the frequency shifted response waves reflected from boundaries within the examination region, to generate ultrasound images. The first set of sensors 108 for the ultrasound probe 101 can include an inertial measurement unit to track the position of the ultrasound probe 101 at the time of an acquired measurement and associate the acquired measurement to the position of the ultrasound probe 101 for reconstructing the examination region of patient 152. In some examples, the reconstruction of the examination region includes a multi-dimensional representation (e.g., 2-dimensional, 3-dimensional) representations of the examination region with associated measurements to describe characteristics of the region e.g., blood flow, tissue density, blood vessel or arterial stiffness, and vessel diameter.

In some implementations, the additional sensors of the ultrasound probe 101 include an inertial measurement unit to track the position of the ultrasound probe 106 and provide error correction if an inaccurate measurement is detected. As an example, the ultrasound system 100 may use position information from the inertial measurement unit to indicate to the user that a measurement needs to be re-recorded and provides the user with an indicator (e.g., visual) of how the new measurement should be performed e.g., by applying additional force or less force in a direction 154A towards or away from the patient.

The ultrasound system 100 can be configured to use position data from an inertial measurement unit in conjunction with other sensor data and perform data fusion to create a volume reconstruction of the examination region of patient 152. The volume reconstruction of the examined region may be created using sensors at the tip of the ultrasound probe 101, but the fidelity of the reconstructed volume can be improved by incorporating additional data from sensors on the first set of sensors 108, the second set of sensors 110, the additional set of sensors 112, or some combination thereof, of the ultrasound probe 101. For example, the inertial measurement unit data may be fused with data from the structured light or camera measurements to improve the accuracy of the ultrasound probe pose e.g., how a user of the ultrasound system 100 or the orientation of the ultrasound probe 101. In some implementations, the pose of an ultrasound probe 101 describes the orientation e.g., angle of approach, position coordinates, representations in space, of the ultrasound probe 101 relative to ultrasound system 100 and the patient 152.

In some implementations, measurements from sensors, e.g., additional sensors 112 can include measurements for forces e.g., torque, shear, and normal forces, which may be used by the ultrasound system 100 to determine an appropriate orientation of the ultrasound probe 101. Improving an orientation of the ultrasound probe 101 improves the quality of measurements achieved by the ultrasound system 100 to provide an accurate diagnosis of medical conditions, e.g., recovery, disease progression.

Referring to FIG. 1A, the additional sensors 112 can include the ultrasound probe 101 can include a structured light source to project a light pattern on the examination region of patient 152. The structured light source can provide depth and surface information of the examination region to the ultrasound system 100, in conjunction with another sensor e.g., a camera system among the additional sensors 112. As an example, the structured light source can be included to improve the accuracy of other indirect contact sensors e.g., cameras, that record visual characteristics of the examination region. The structured light source may provide a surface geometry measurement from the ultrasound probe body 106, while another sensor at the tip of the ultrasound probe 101 records additional data by making contact with the examination region of patient 152.

In another example, sensors mounted on the ultrasound probe 101 can include a scanning laser. A scanning laser can provide a form of visual user feedback to show the user how to adjust the orientation of the ultrasound probe 101 when acquiring data. For example, the scanning laser may create a visual aid, e.g., a signal in the shape of an arrow pointing in a direction, to indicate to the user to move the ultrasound probe 101 in a direction such as direction 154B, e.g., in a transverse direction along the surface of a patient's appendage. For example, the scanning laser may create a visual aid, e.g., a status bar or map on the subject surface e.g., skin of patient 152, to indicate areas that need additional data collection.

As an example, the additional sensors for the ultrasound probe 101 can include one or more cameras. The one or more cameras can be configured to provide images to calibrate the first set of sensors 108, e.g., referring to FIG. 1A above. The one or more cameras can also be utilized to estimate a surface geometry of the examination region. The one or more camera sensors of the ultrasound probe 101 can operate while the ultrasound probe 101 captures ultrasound image, e.g., to provide a mapping between surface geometry measurements from the camera and internal geometry measurements provided by the ultrasound system 100. The mapping between surface and internal geometry measurements can be utilized, e.g., by the computing device 120, for enhanced processing to build a multi-dimensional model of the examination region for further diagnosis or rehabilitation and recovery.

The additional sensors 112 of the ultrasound probe 101 can also include capacitive sensors to measure changes in electrical field detected by the capacitive sensor. For example, the capacitive sensors may measure the electrical properties of the skin in the examination region of the patient 152 to describe dielectric properties of the examination region. In some implementations, the capacitive sensor measures dielectric properties e.g., of muscle tissue. The capacitive sensors may include high-resolution measurements of near surface structures (e.g., fluid or layers of tissue) underneath the surface of skin of the examination region. The capacitive sensors may include measurements to describe air gaps beneath one or more other sensors at the tip of the ultrasound probe 101.

In some implementations, the ultrasound probe 101 includes additional sensors at the tip of the ultrasound probe to make direct contact with the examination region of patient 152. As an example, the sensors at the tip of the ultrasound probe 101 can include a skin resistance sensor. The skin resistance sensor can describe the state of the skin of the examination region of patient 152, e.g., the dryness or moisture level of the skin. The skin resistance sensor can describe how well the skin of the examination region of patient 152 is lubricated, and can include electrical properties of the skin e.g., dielectric properties including conductivity, resistivity, thermoelectricity, and temperature coefficient of resistance.

In some implementations, the additional sensors 112 of the ultrasound probe 101 can include a pressure sensor, a force sensor, or some combination therein. A force sensor at the tip of the ultrasound probe 101 can measure forces including torque and shear force, which the ultrasound system 100 can utilize to determine derived forces including friction. For example, a measurement of friction derived from shear and torque forces can help a user determine how well lubricated the skin of the examination region, and if adjustments to lubrication are required for improved measurement quality. A force sensor at the tip of the ultrasound probe 101 may also measure normal forces to determine if enough contact between the ultrasound probe 101 and the examination region of the patient 152 is established for measurement of the examination region. In some examples, the force sensor measures a stiffness of tissue over a time period, e.g., by repeatedly measuring the examination region of patient 152 with the ultrasound system 100. A pressure sensor may be used to determine a distribution of force e.g., a pressure field across the examination region of patient 152.

In some implementations, the additional sensors 112 can include a sensor at the tip of the ultrasound probe 101 that is a photoplethysmography (PPG) sensor to monitor heart rate and blood pressure of the patient 152 at the examination region. The ultrasound probe 101 can include the PPG sensor and record pulse waveforms emitted by the sensor, which can then be correlated to measurements recorded by the ultrasound probe 101, providing an additional characteristic or dimensionality to a volume reconstruction of the examination region. For example, the PPG sensor may provide pulse wave information correlated to ultrasound measurements from the ultrasound probe 101 that when processed, may provide a blood velocity map of the examination region of the patient 152 as the pulse travels through the patient 152. The PPG sensor may be coupled with a pulse oximeter sensor that provides a mechanical measurement for the pulse wave from the PPG sensor.

In some implementations, the handle 102 of the ultrasound probe 101 may include feedback devices that provide user feedback by generating audible noise e.g., buzzing or pinging, visual feedback on a display or through projected images on a subject surface e.g., the skin of patient 152, or tactile feedback by generating physical forces e.g., vibrations or tumbling, to correct the user operating the ultrasound probe 101. For example, the handle 102 can generate a pinging sound when a successful measurement is acquired by the ultrasound probe 101 to indicate that the user may apply the ultrasound probe 101 to a different examination region of the patient 152 e.g., in a transverse direction 154B. In another example, the handle 102 may project arrows on a surface e.g., skin, of the patient 152 to indicate the preferred scan direction e.g., directions 154B or areas that need more data collection. In another example, the handle 102 can generate a vibration when a poor measurement is acquired by the ultrasound probe 101 to indicate to the user to apply additional pressure by pressing the ultrasound probe 101 towards the examination region of the patient 152 e.g., in an inward direction 154A.

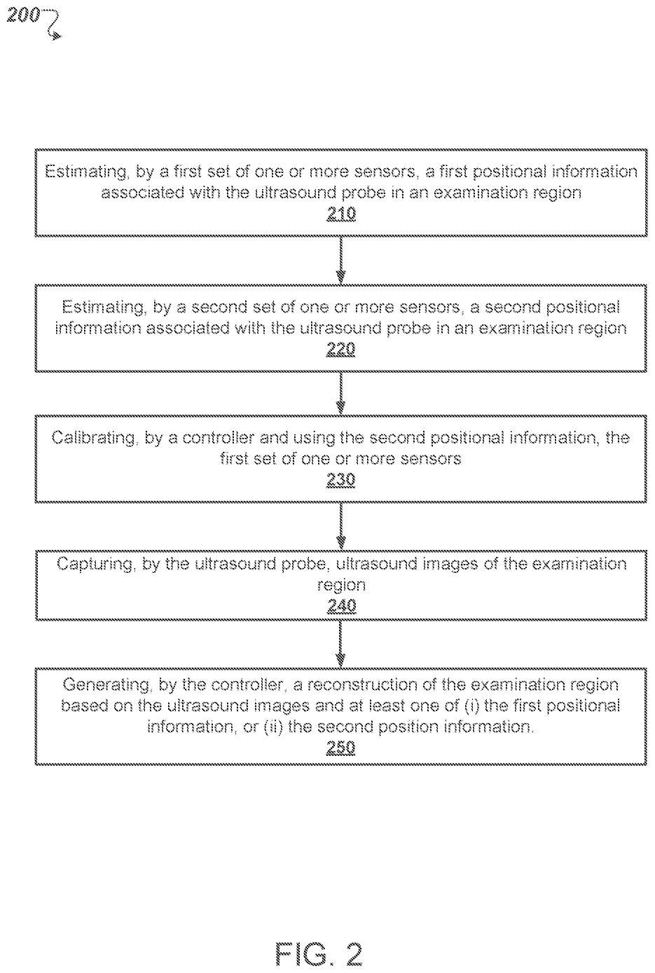

FIG. 2 is a flow diagram showing an example of a process 200 for calibrating a first set of sensors of an ultrasound system using a second set of sensors of the ultrasound system. The process 200 can be performed by one or more systems that include the controller 114, the computing device 120, or some combination therein.

The process 200 includes estimating, by a first set of one or more sensors coupled to an ultrasound probe, a first positional information associated with the ultrasound probe in an examination region (210). The first set of one or more sensors can include the first set of sensors 108 described in reference to FIG. 1A above. Examples of a sensor from the set of one or more sensors can include mechanical-based sensors, including gyroscopes, gimbals, and related components, e.g., inertial measurement units. The first positional information can include pose information, such as a position, an orientation, or some combination thereof, of the ultrasound probe.

The process 200 includes estimating, by a second set of one or more sensors coupled to the ultrasound probe, a second positional information associated with the ultrasound probe, wherein the second set of one or more sensors is configured to capture electromagnetic force (EMF) measurements in the examination region (220). The second positional information can be estimated from measurements captured by an EMF detector. In some implementations, additional data of the examination region can be captured by an additional set of one or more sensors.

The process 200 includes calibrating, by a controller and using the second position information, the first set of one or more sensors (230). Examples of the controller can include the controller 114 described in reference to FIG. 1A above. The controller can be configured to calibrate by the first set of one or more sensors by adjusting configurations of components of the sensor, compensating the measurements output by the sensor, adjusting a capture rate of measurements for the sensor, or some combination thereof. Calibrating the first set of one or more sensors can include a difference between the first positional information and the second positional information.

The process 200 includes capturing, by the ultrasound probe, ultrasound images of the examination region (240). The ultrasound images of the examination region are captured while the first set of one or more sensors is calibrated. In some implementations, the first set of one or more sensors is calibrated when sensor measurements are within a threshold value of a known position measurement.

The process 200 includes generating, by at least one of (i) a computing device, or the controller, a reconstruction of the examination region based on the ultrasound images and at least one of (i) the first positional information, or (ii) the second position information (250). In some implementations, the reconstruction of the examination region can be generated based on additional data provided by an additional set of one or more sensors.

In some implementations, the process 200 includes a model processing the ultrasound images and at least one of (i) the first positional information, or (ii) the second positional information, to generate a reconstruction of the examination region. The model can also generate a set of surface measurements associated with the examination region. In some implementations, the model is a machine learning network, a physical simulation, a computational model, or some combination thereof. The set of surface measurements can include vessel stiffness, vessel diameter, and other types of surface data for the examination region.

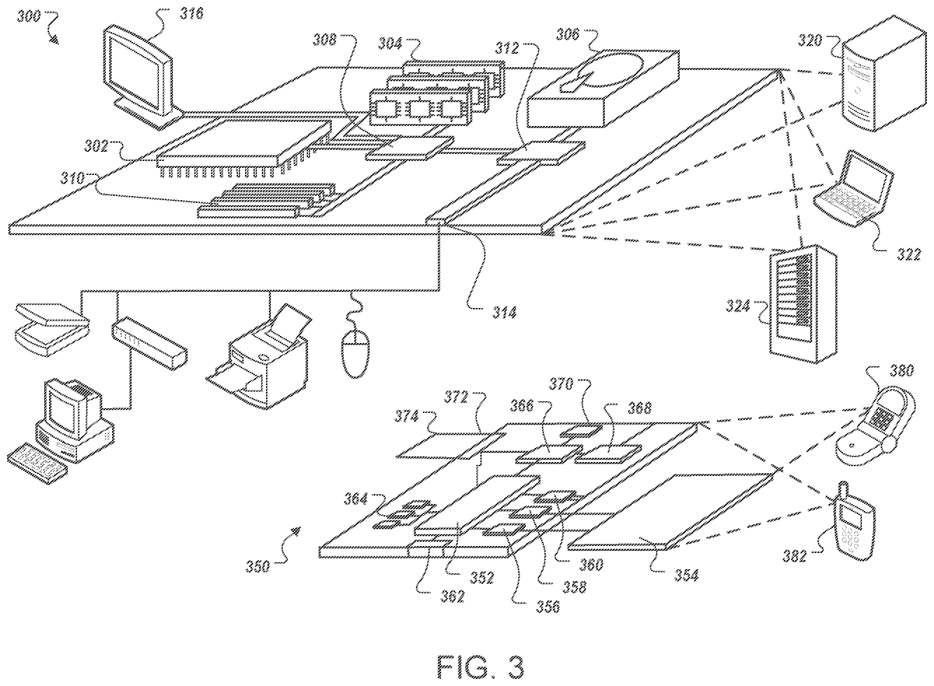

FIG. 3 is a diagram illustrating an example of a computing system used for calibrating sensors of an ultrasound system, e.g., computing device 120, controller 114. The computing system includes computing device 300 and a mobile computing device 350 that can be used to implement the techniques described herein. For example, one or more components of the controller 114 and the computing device 120 is an example of the computing device 300 or the mobile computing device 350.

The computing device 300 is intended to represent various forms of digital computers, such as laptops, desktops, workstations, personal digital assistants, servers, blade servers, mainframes, and other appropriate computers. The mobile computing device 350 is intended to represent various forms of mobile devices, such as personal digital assistants, cellular telephones, smart-phones, mobile embedded radio systems, radio diagnostic computing devices, and other similar computing devices. The components shown here, their connections and relationships, and their functions, are meant to be examples only and are not meant to be limiting.

The computing device 300 includes a processor 302, a memory 304, a storage device 306, a high-speed interface 308 connecting to the memory 304 and multiple high-speed expansion ports 310, and a low-speed interface 312 connecting to a low-speed expansion port 314 and the storage device 306. Each of the processor 302, the memory 304, the storage device 306, the high-speed interface 308, the high-speed expansion ports 310, and the low-speed interface 312, are interconnected using various buses, and may be mounted on a common motherboard or in other manners as appropriate. The processor 302 can process instructions for execution within the computing device 300, including instructions stored in the memory 304 or on the storage device 306 to display graphical information for a Graphical User Interface (GUI) on an external input/output device, such as a display 316 coupled to the high-speed interface 308. In other implementations, multiple processors and/or multiple buses may be used, as appropriate, along with multiple memories and types of memory. In addition, multiple computing devices may be connected, with each device providing portions of the operations (e.g., as a server bank, a group of blade servers, or a multi-processor system). In some implementations, the processor 302 is a single threaded processor. In some implementations, the processor 302 is a multi-threaded processor. In some implementations, the processor 302 is a quantum computer.

The memory 304 stores information within the computing device 300. In some implementations, the memory 304 is a volatile memory unit or units. In some implementations, the memory 304 is a non-volatile memory unit or units. The memory 304 may also be another form of computer-readable medium, such as a magnetic or optical disk.

The storage device 306 is capable of providing mass storage for the computing device 300. In some implementations, the storage device 306 may be or include a computer-readable medium, such as a floppy disk device, a hard disk device, an optical disk device, or a tape device, a flash memory or other similar solid-state memory device, or an array of devices, including devices in a storage area network or other configurations. Instructions can be stored in an information carrier. The instructions, when executed by one or more processing devices (for example, processor 302), perform one or more methods, such as those described above. The instructions can also be stored by one or more storage devices such as computer- or machine-readable mediums (for example, the memory 304, the storage device 306, or memory on the processor 302). The high-speed interface 308 manages bandwidth-intensive operations for the computing device 300, while the low-speed interface 312 manages lower bandwidth-intensive operations. Such allocation of functions is an example only. In some implementations, the high speed interface 308 is coupled to the memory 304, the display 316 (e.g., through a graphics processor or accelerator), and to the high-speed expansion ports 310, which may accept various expansion cards (not shown). In the implementation, the low-speed interface 312 is coupled to the storage device 306 and the low-speed expansion port 314. The low-speed expansion port 314, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 300 may be implemented in a number of different forms, as shown in the figure. For example, it may be implemented as a standard server 320, or multiple times in a group of such servers. In addition, it may be implemented in a personal computer such as a laptop computer 322. It may also be implemented as part of a rack server system 324. Alternatively, components from the computing device 300 may be combined with other components in a mobile device, such as a mobile computing device 350. Each of such devices may include one or more of the computing device 300 and the mobile computing device 350, and an entire system may be made up of multiple computing devices communicating with each other.

The mobile computing device 350 includes a processor 352, a memory 364, an input/output device such as a display 354, a communication interface 366, and a transceiver 368, among other components. The mobile computing device 350 may also be provided with a storage device, such as a micro-drive or other device, to provide additional storage. Each of the processor 352, the memory 364, the display 354, the communication interface 366, and the transceiver 368, are interconnected using various buses, and several of the components may be mounted on a common motherboard or in other manners as appropriate.

The processor 352 can execute instructions within the mobile computing device 350, including instructions stored in the memory 364. The processor 352 may be implemented as a chipset of chips that include separate and multiple analog and digital processors. The processor 352 may provide, for example, for coordination of the other components of the mobile computing device 350, such as control of user interfaces, applications run by the mobile computing device 350, and wireless communication by the mobile computing device 350.

The processor 352 may communicate with a user through a control interface 358 and a display interface 356 coupled to the display 354. The display 354 may be, for example, a TFT (Thin-Film-Transistor Liquid Crystal Display) display or an OLED (Organic Light Emitting Diode) display, or other appropriate display technology. The display interface 356 may include appropriate circuitry for driving the display 354 to present graphical and other information to a user. The control interface 358 may receive commands from a user and convert them for submission to the processor 352. In addition, an external interface 362 may provide communication with the processor 352, so as to enable near area communication of the mobile computing device 350 with other devices. The external interface 362 may provide, for example, for wired communication in some implementations, or for wireless communication in other implementations, and multiple interfaces may also be used.

The memory 364 stores information within the mobile computing device 350. The memory 364 can be implemented as one or more of a computer-readable medium or media, a volatile memory unit or units, or a non-volatile memory unit or units. An expansion memory 374 may also be provided and connected to the mobile computing device 350 through an expansion interface 372, which may include, for example, a SIMM (Single In Line Memory Module) card interface. The expansion memory 374 may provide extra storage space for the mobile computing device 350, or may also store applications or other information for the mobile computing device 350. Specifically, the expansion memory 374 may include instructions to carry out or supplement the processes described above, and may include secure information also. Thus, for example, the expansion memory 374 may be provided as a security module for the mobile computing device 350, and may be programmed with instructions that permit secure use of the mobile computing device 350. In addition, secure applications may be provided via the SIMM cards, along with additional information, such as placing identifying information on the SIMM card in a non-hackable manner.

The memory may include, for example, flash memory and/or NVRAM memory (nonvolatile random access memory). In some implementations, instructions are stored in an information carrier such that the instructions, when executed by one or more processing devices (e.g., processor 352), perform one or more methods, such as those described above. The instructions can also be stored by one or more storage devices, such as one or more computer or machine-readable mediums (for example, the memory 364, the expansion memory 374, or memory on the processor 352). In some implementations, the instructions can be received in a propagated signal, for example, over the transceiver 368 or the external interface 362.

The mobile computing device 350 may communicate wirelessly through the communication interface 366, which may include digital signal processing circuitry in some cases. The communication interface 366 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), LTE, 3G/4G cellular, among others. Such communication may occur, for example, through the transceiver 368 using a radio frequency. In addition, short-range communication may occur, such as using a Bluetooth, Wi-Fi, or other such transceiver (not shown). In addition, a GPS (Global Positioning System) receiver module 370 may provide additional navigation- and location-related wireless data to the mobile computing device 350, which may be used as appropriate by applications running on the mobile computing device 350.

The mobile computing device 350 may also communicate audibly using an audio codec 360, which may receive spoken information from a user and convert it to usable digital information. The audio codec 360 may likewise generate audible sound for a user, such as through a speaker, e.g., in a handset of the mobile computing device 350. Such sound may include sound from voice telephone calls, may include recorded sound (e.g., voice messages, music files, among others) and may also include sound generated by applications operating on the mobile computing device 350.

The mobile computing device 350 may be implemented in a number of different forms, as shown in the figure. For example, it may be implemented as a cellular telephone 380. It may also be implemented as part of a smart-phone 382, personal digital assistant, or other similar mobile device.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. An ultrasound system, comprising:
an ultrasound probe configured to capture ultrasound images of an examination region;
a first set of one or more sensors coupled to the ultrasound probe and configured to estimate a first positional information associated with the ultrasound probe;
a second set of one or more sensors coupled to the ultrasound probe and configured to capture electromagnetic force (EMF) measurements in the examination region to estimate a second positional information associated with the ultrasound probe, wherein second positional information is used to calibrate the first set of one or more sensors; and
a controller configured to use at least one of (i) the first positional information, or (ii) the second positional information to generate a reconstruction of the examination region based on ultrasound images captured by the ultrasound probe.

2. The ultrasound system of claim 1, wherein the first positional information comprises at least one of (i) a position, or (ii) an orientation, of the ultrasound probe.

3. The ultrasound system of claim 1, wherein the second positional information comprises at least one of (i) a position, or (ii) an orientation, of the ultrasound probe.

4. The ultrasound system of claim 1, wherein calibrating the first set of one or more sensors comprises adjusting a capture rate of at least one of (i) ultrasound images from the ultrasound probe, (ii) a sensor from the first set of one or more sensors, or (iii) a sensor from the second set of one or more sensors, based on at least one of (i) the first positional information, or (ii) the second positional information.

5. The ultrasound system of claim 1, wherein a sensor in the first set of one or more sensors comprises a gimbal or a gyroscope.

6. The ultrasound system of claim 1, comprising:
a computing device communicatively coupled to the controller, wherein the computing device is configured to perform operations comprising:
processing, by a model, the ultrasound images and at least one of (i) the first positional information, or (ii) the second positional information; and
generating, by the model, the reconstruction of the examination region and a set of surface measurements associated with the examination region.

7. The ultrasound system of claim 6, wherein the set of surface measurements comprises at least one of (i) vessel stiffness, or (ii) vessel diameter, of the examination region.

8. The ultrasound system of claim 6, wherein the model is at least one of (i) a machine learning network, (ii) a physical simulation, or (iii) a computational model.

9. The ultrasound system of claim 1, comprising:
an additional set of one of more sensors coupled to the ultrasound probe, the additional set of one or more sensors configured to capture additional data of the examination region;
wherein the controller is configured to generate the reconstruction of the examination region based on the ultrasound images and the additional data.

10. The ultrasound system of claim 9, wherein a sensor in the additional set of one or more sensors comprises a pressure sensor configured to capture force measurements of the examination region.

11. The ultrasound system of claim 9, wherein a sensor in the additional set of one or more sensors comprises a camera device configured to capture one or more images of the examination region.

12. The ultrasound system of claim 11, wherein a second sensor in the additional set of sensors comprises
a structured light source configured to illuminate at least a portion of the examination region;
wherein the camera device is configured to capture one or more images of the examination region while the portion of the examination region is illuminated.

13. The ultrasound system of claim 9, wherein the additional set of one or more sensors comprises a plurality of peripheral sensors, wherein a peripheral sensor from the plurality of peripheral sensors is configured to capture surface measurements of the examination region.

14. The ultrasound system of claim 13, wherein the peripheral sensor is at least one of:
(i) a skin resistance sensor configured to capture skin resistivity measurements of the examination region,
(ii) a pulse oximeter configured to capture pulse rate measurements of the examination region, or
(iii) a photoplethysmography sensor configured to capture at least one of (i) pulse rate measurement or (ii) blood pressure measurement of the examination region.

15. The ultrasound system of claim 13, wherein the plurality of peripheral sensors comprises:
a light emitting diode configured to emit a pattern of light on at least a portion of the examination region; and
a light detector configured to determine a deformation of the pattern of light on the portion of the examination region;
wherein the additional data of the peripheral sensor comprises depth measurements representing the deformation of the portion of the examination region.

16. The ultrasound system of claim 1, wherein:
the second set of one or more sensors comprises a pair of sensors;
a first sensor in the pair of sensors is positioned onto a surface of the examination region;
a second sensor in the pair of sensors is mounted onto the ultrasound probe; and
the controller is configured to determine an amount of distortion based on a first set of EMF measurements from the first sensor and a second set of EMF measurements from the second sensor.

17. A computer-implemented method, the method comprising:
estimating, by a first set of one or more sensors coupled to an ultrasound probe, a first positional information associated with the ultrasound probe in an examination region;
estimating, by a second set of one or more sensors coupled to the ultrasound probe, a second positional information associated with the ultrasound probe, wherein the second set of one or more sensors is configured to capture electromagnetic force (EMF) measurements in the examination region;
calibrating, by a controller and using the second position information, the first set of one or more sensors;
capturing, by the ultrasound probe, ultrasound images of the examination region; and
generating, by the controller, a reconstruction of the examination region based on the ultrasound images and at least one of (i) the first positional information, or (ii) the second position information.

18. The computer-implemented method of claim 17, comprising:
capturing, by an additional set of one or more sensors, additional data of the examination region;
generating, by the controller, the reconstruction of the examination region based on the ultrasound images and the additional data.

19. The computer-implemented method of claim 17, wherein calibrating the first set of one or more sensors comprises adjusting a capture rate of at least one of (i) ultrasound images from the ultrasound probe, or (ii) positional information from a sensor in the first set of one or more sensors.

20. The computer-implemented method of claim 17, wherein a sensor in the first set of one or more sensors comprises a gimbal or a gyroscope.

21. The computer-implemented method of claim 17, comprising:
processing, by a model, the ultrasound images and at least one of (i) the first positional information, or (ii) the second positional information; and
generating, by the model, the reconstruction of the examination region and a set of surface measurements associated with the examination region.

22. The computer-implemented method of claim 17, wherein calibrating the first set of one or more sensors comprises determining a difference between the first positional information and the second positional information.

23. A non-transitory, computer-readable medium storing one or more instructions executable by a computer system to perform operations comprising:
estimating, by a first set of one or more sensors coupled to an ultrasound probe, a first positional information associated with the ultrasound probe in an examination region;
estimating, by a second set of one or more sensors coupled to the ultrasound probe, a second positional information associated with the ultrasound probe, wherein the second set of one or more sensors is configured to capture electromagnetic force (EMF) measurements in the examination region;
calibrating, by a controller and using the second position information, the first set of one or more sensors;
capturing, by the ultrasound probe, ultrasound images of the examination region; and
generating, by the controller, a reconstruction of the examination region based on the ultrasound images and at least one of (i) the first positional information, or (ii) the second position information.
